(19) United States
(12) Patent Application Publication
(10) Pub. No.: US 2024/0307274 A1
(43) Pub. Date: Sep. 19, 2024

(54) CONCENTRATED RECONSTITUTABLE LIQUID COMPOSITIONS FOR PERSONAL CARE

(71) Applicant: GALAXY SURFACTANTS LTD., Navi Mumbai (IN)

(72) Inventors: Nirmal Koshti, Piscataway, NJ (US); Bhagyesh Jagannath Sawant, Kalyan (E) (IN); Amol Gajanan Jadhav, Dombivli (E) (IN); Parag Narendra Savla, Bhiwandi (IN)

(21) Appl. No.: 18/441,107

(22) Filed: Feb. 14, 2024

(30) Foreign Application Priority Data
Mar. 2, 2023 (IN) 202321014089

(51) Int. Cl. A61K 8/02; A61Q 5/02; A61Q 19/10

(52) U.S. Cl. CPC A61K 8/0204; A61Q 5/02; A61Q 19/10; A61K 2800/30; A61K 2800/524; A61K 2800/596; A61K 2800/805

(57) ABSTRACT

A concentrated reconstitutable liquid compositions for personal care and a process of making thereof. The composition comprises a blend of sodium lauroyl methyl taurate and sodium lauroyl methyl isethionate in a ratio of 1:2 with solid content of greater than 40%; b. cocamidopropyl betaine with solid content of greater than 40%; c. an antimicrobial preservative; and d. cosmetically and pharmaceutically acceptable auxiliaries. The compositions are low-viscous ‘easy-to-transfer’ and ‘easy-to-hydrate’ and upon dilution with water, afford high viscous ‘ready-to-use’ personal cleansing compositions such as hand-washes, body-washes or shampoos. The liquid compositions of the present invention are based on surfactants only and are free of thickeners such as natural gums or synthetic polymers or other ‘structuring agents’.

### FIELD OF THE INVENTION

The present invention relates to concentrated, hydratable, and reconstitutable liquid compositions for personal cleansing wherein the end-formulations are made by the consumer by diluting the same with water in a reusable refill container. These concentrated compositions, upon dissolution with water followed by gentle shaking, transform into end-wash formulations like bodywash, shampoo etc. with customary viscosity and with pH that is close to biological pH of skin.

### BACKGROUND AND THE PRIOR ART OF THE INVENTION

The concept of converting concentrated and hydratable liquid surfactants-mix into ‘ready-to-use’ body washes by adding it to requisite quantity of water, has been around for last couple of years (Hiban et al. US 2021/0220243). This is an extremely useful and practical concept directed towards drastic reduction of use of plastic by encouraging consumers to use ‘do-it-by-themselves’ the cleansing compositions for personal hygiene. In addition to restricting plastic consumption, a significant saving of energy is achieved in terms of transportation when consumers use concentrated compositions and reconstitute them by simply adding to definite amount of water and shaking gently in a refill container to get the final personal cleansing formulation. The example of concentrated liquid product in the market is from Unilever's Dove body wash where concentrated liquid of lower viscosity (for complete transfer into refill container) is emptied into a refill bottle containing water (three times by weight of concentrated liquid) and shaken to get a uniform liquid of higher viscosity which is customary for a consumer use.

With global warming getting out-of-control, every effort by a consumer is welcome that contributes towards achieving the tall goals that are designed to stop and reverse the process of global warming. The concept of concentrated and reconstitutable compositions for personal care addresses and contributes towards saving on energy, significant reduction in usage of non-degradable material, burning less fossil fuel for road transportation (reduction in greenhouse gases) etc. US 2021/0220243 A1 (Hiban et al.) discloses hydratable concentrated surfactant composition. The composition is reported to be low viscous and in lamellar phase. Upon dilution it is reported to go to an isotropic phase with increase in viscosity. The concentrated composition is diluted by the consumer in a refill bottle and thus significant reduction in plastic usage. The composition of US published application 2021/0220243 A1 is made up of surfactants (anionic and amphoteric/zwitterionic) and a structuring agent. The key additive disclosed in US published application 2021/0220243 A1 is the structuring agent, a C6-C14 alcohol or a C6-C14 fatty acid which is present in significant percentage of the concentrated form of the composition.

All thirty-four examples of US 2021/0220243 A1 are reported with lauric acid as the structuring agent ranging from 4.6% to 11.2% by weight of the total composition (10 to 30% of total surfactants in the composition, in Example 1, lauric acid is 12% of total surfactants and in Example 5 has 23% lauric acid of the total surfactants), and in many examples, lauric acid (the structuring agent) is used in higher percentages than the anionic surfactants, mainly acyl isethionate and additionally, an acyl taurate or an acyl glycinate. The concentrated compositions of this patent application are reported with anionic surfactants with zwitterionic surfactant, cocamidopropyl betaine (CAPB) wherein the latter is higher percentage than the anionic surfactants, typically the ratio is 3:1 by weight (betaine: anionic surfactant). The patent application reveals that with this kind of ratio for zwitterionic surfactant to anionic surfactants, the structuring agent needs to be over 15% of total surfactant present in the composition. If the ratio zwitterionic surfactant to anionic surfactant is 1.5 or below, then lauric acid (the structuring agent) needs to be around 27% of the total surfactant concentration. Thus, zwitterionic surfactant content is always higher than the anionic surfactants. Examples 8 to 13 of this patent application show that if cocamindopropyl betaine is lowered to the level of anionic surfactants present then the concentrated compositions turn extremely viscous and on dilution the resultant viscosity is too low to be customary personal care cleansers.

It should be noted that typically, in gentle skin cleansers, anionic surfactants (like acyl isethionate and acyl amino acid surfactants) are used in higher % than zwitterionic cocamidopropyl betaine. Acyl isethionates and amino acid surfactants are mild toward both proteins and lipids of the stratum corneum (SC) whereas CAPB is mild toward proteins of SC, but it has very strong solvating effect on the lipids of SC, thus weakening the barrier function of SC. This leads to increase in trans epidermal water loss (TEWL) resulting into dry skin.

US 2021/0220243 A1 discloses the pH range of end-use compositions from 4.5 to 10. At such a high alkaline pH (pH 9 or pH 10), lauric acid (the structuring agent used in all the thirty-four examples) will exist (significant %) as sodium laurate which is nothing but soap. Soap with alkaline pH is not suitable for personal care cleansing like body wash or shampoo. Cleansing with soaps of alkaline pH leads to substantial drying of skin (soap removes lipids from stratum corneum, thus resulting into increase in trans-epidermal water loss). Also, alkaline pH swells the proteins (keratin bundles) of stratum corneum resulting in weakening of skin's barrier function and leading to higher rate of water loss. The advantage of gentle cleansing with the mild surfactants, is lost if the pH is on the alkaline side for the skin cleansers. Skin's natural pH is around 5.5 and is favorable for the natural skin microbiota that does a job of protecting skin from invading pathogenic microbes. This need of creating right ratio of primary surfactants (anionic) to secondary surfactants (zwitterionic) wherein typically the former is higher concentration that the latter, has been addressed by Indian Patent application IN 202221026146). This has been achieved by generating mixtures of dry surfactants (powder form) in the desired ratio so that dilution of the powder-blend with water yields viscous flowable end-use composition with skin pH and transparency. These solid compositions of Indian pat appl. IN 202221026146 are based on surfactants only and don't contain any additive or structuring agent or rheology modifier.

The biggest disadvantage of these concentrated surfactant blends in powder form (or any other solid format) is the cost of drying the surfactants. All anionic surfactants like sodium acyl taurates, sodium acyl glycinate or glutamates, sulphosuccinates are produced as aqueous solutions of, typically, 30% active (from corresponding fatty acid chloride and amino acid in the presence of alkali). Most anionic surfactants are preferably made in water medium since they are alkali salts of either carboxylic acids or sulphonic acids or phosphonic acid. The same is true with zwitterionic betaines which are ionic, and the by-products are ionic inorganic salts that are soluble in water. Acylation of amino acids by acyl chloride is done at milder conditions in aqueous medium and hence yields purer products compared to heating of fatty acids with amino acids at temperatures close to 200° C.

The dilute solutions of ‘sulphate-free’ anionic surfactants (typically 30% solids) need to be dried for making powder or any other solid form. Evaporating all (60-70%) water by operations like spray drying etc. is extremely expensive operation. In addition to being energy intensive drying operation (evaporation of 60-70% of water), another serious concern is dusting of dried surfactants and its adverse impact on human personnel due to inhalation of the dust. The dust of surfactants also come with the ‘dust explosion hazard’ (https://www.osha.gov/combustible-dust) necessitating special manufacturing set-up to eliminate the possibility of ignition of dust by any static electricity generation. Drying of cocamidopropyl betaine, zwitterionic surfactant, needs ‘corrosion-resistant’ equipment since the pH is acidic and it contains significant level of sodium chloride. The spray dried powder of CAPB is extremely hygroscopic and hence handling it on a bulk scale is not easy. Mixing dried surfactants with different particles sizes and different flow properties and with various degree of hygroscopicity is challenging. The powder form of surfactants is hazardous due to the dust explosion properties of powder. Producing surfactants in aqueous medium and then drying by expensive process (evaporation of 70% of water) and then reconstituting the dry powder with water again doesn't make sense in long term sustainability. The powder compositions disclosed in IN 202221026146 don't give any special advantage in terms of higher degree of dilution with water.

On the backdrop of these serious limitations of the prior art which uses structuring aid for the concentrated liquid surfactants (US 2021/0220243 A1) or dry (powder or tablet) format (IN 202221026146) that not only allows on solid ingredients in the dry composition, but solid formats are fraught with serious issues of ‘dust inhalation’ and ‘dust explosion’, there is a definite need to produce concentrated, ‘easy-to-reconstitute’, ‘sulphate-free’ surfactant systems that would meet the criteria for consumer friendly ‘DIY’ concept (Do-it-Yourself) enabling significant saving on the usage of plastic, conservation of water and energy and less generation of greenhouse gases. However, the serious limitations of the solid formats (restriction on ingredients, powder format is too difficult to handle by the DIY type of consumers due to dusting, dust explosion while dealing with powder manufacture on bulk scale) needs to be noted. Compacting powder into tablet becomes another additional step adding to the cost of drying the surfactants. Unexpectedly, the inventors of present invention have found ways of making concentrated aqueous anionic ‘sulphate-free’ surfactants that can be combined with concentrated aqueous zwitterionic surfactant in the right ratio (zwitterionic surfactant lesser than anionic like acyl isethionates and acyl amino-acid surfactants) to afford low-viscous, concentrated composition without any structuring aid which upon dilution with water results in end-wash formulations with customary viscosity and desired aesthetics.

### Objectives of the Invention:

It is an objective of the present invention to create concentrated yet user-friendly (easy-to-pour, low viscous), compositions suitable for making personal care cleansing compositions without the drawbacks of the prior art.

It is an objective of the present invention is to create concentrated reconstitutable compositions of surfactants, which upon dilution with water by the consumer, would afford the personal care cleansing formulations in a reusable refill-container. This repeat use of refill-container significantly reduces the plastic consumption, less water consumption and less generation of greenhouse gases (transportation of concentrated products as against the dilute ‘finished’ or ‘end-use’ wash products in market).

Yet another objective of the present invention is to create the concentrated and reconstitutable liquid compositions of ‘sulphate-free’ surfactants without any formulation aids like stabilizing or thickening or structuring agents. The objective is to create concentrated reconstitutable compositions which upon dissolution with water would thicken without aid of any structuring agent like fatty alcohol or fatty acids or a natural or synthetic polymeric gelling or thickening agent.

Another objective of the present patent application is to create concentrated yet ‘easy-to-pour’ compositions that would obviate the need for the dry form of concentrated surfactants.

Yet another objective of the current invention is to create low viscous ‘sulphate-free’ surfactants concentrate that can accommodate the desired personal care actives and transform into end-compositions with customary viscous nature upon dilution with water.

Yet another objective of the present invention is to create ‘easy-to-pour’, concentrated and reconstitutable compositions that would afford a variety of personal cleansing compositions like shampoo, bodywash, facewash and handwash by varying the degree of reconstituting (varying the extent of dilution) of the concentrated composition with water.

Yet another objective of present invention is to create ‘easy-to-pour’, concentrated and reconstitutable compositions that, upon dilution with water, would afford a variety of personal cleansing composition like shampoo, body wash, face wash and handwash with superior mild cleansing performance at pH that is close to natural pH of skin (biological pH of 5.0 to 6.0)

It is another objective of the present invention is to create concentrated and ‘easy-to-reconstitute’ compositions which upon dissolution in water would afford the personal care formulations like handwash, bodywash and shampoo without deploying harsh surfactants like alkyl sulphate or ether sulphates and without compromising on superlative performance in terms of lather and gentleness at biological pH of skin.

### SUMMARY OF THE INVENTION

Accordingly, the present invention provides a concentrated reconstitutable liquid composition comprising of:
a. blend of sodium lauroyl methyl taurate and sodium lauroyl methyl isethionate, in the molar ratio of 1:2 and with solids content of minimum 40%;
b. cocamidopropyl betaine with solids content of minimum 40%;
c. 1.0% to 2.5% by weight of total composition, an antimicrobial preservative;
d. 0.5% to 5% by weight of total composition, a cosmetically and pharmaceutically acceptable auxiliaries; and
wherein, the blend of sodium lauroyl methyl taurate and sodium lauroyl methyl isethionate is synthesized using lauroyl chloride;
wherein the ratio of a: b:: 5.0 to 6.0: 3.5 to 4.5 by weight; and the said composition is without any additionally added fatty alcohol or fatty acid as a structurant;
wherein the said composition when diluted 3-5 times, by weight with water to afford a liquid cleansing formulation with pH ranging from 5.0 to 6.0 and viscosity ranging from 2,000 to 10,000 cps at 25° C.

The present invention also provides a process to make a reconstitutable concentrated liquid composition comprising:
a. an aqueous blend of sodium lauroyl methyl taurate and sodium lauroyl methyl isethionate;
b. an aqueous solution of cocamidopropyl betaine;
c. 1.0% to 2.5% by weight of total composition, an antimicrobial preservative;
d. 0.5% to 5% by weight of total composition, a cosmetically and pharmaceutically acceptable auxiliaries; and
wherein, the blend of sodium lauroyl methyl taurate and sodium lauroyl methyl isethionate is synthesized using lauroyl chloride in a single reaction vessel;
wherein the ratio of sodium lauroyl methyl taurate and sodium lauroyl methyl isethionate is 1:2;
wherein the solid content of the component a. and the component b. is greater than 40%;
wherein the ratio of a: b:: 5.0 to 6.0: 3.5 to 4.5 by weight; and
wherein the said composition when diluted 3-5 times, by weight, with water, affords a liquid cleansing formulation with pH ranging from 5.0 to 6.0 and viscosity ranging from 2,000 to 10,000 cps at 25° C. wherein the process comprises the steps of:
i) preparing a 40% to 45% solid content blend of sodium lauroyl methyl taurate and sodium lauroyl methyl isethionate;
ii) preparing cocamidopropyl betaine with solid content of 40% to 45% without the aid of any structurant; and
iii) mixing the components of step i) and step ii) along with an antimicrobial preservative and a cosmetically or pharmaceutically auxiliaries.

The present invention provides a process which is specifically designed to render the blend of anionic surfactants in a high concentration and final concentrate having minimum 45% solid without the use of any structurant.

The liquid end-use formulation is a personal cleansing wash like a facewash, a bodywash or a shampoo and is made by consumer by adding water in refill-container that can be used lifelong. The present invention is directed towards significant reduction of plastic, conservation of water and energy and reduction greenhouse gases by transporting and packaging almost one fourth or one fifth of total volume personal wash products.

The above-described features and the advantages of the present disclosures will be appreciated and understood by those skilled in the art from the ‘detailed descriptions’ and ‘the claims’.

### FIGURE DESCRIPTION OF FIGURES

FIG. 1: Depicts ¹H NMR of sodium 2-hydroxy propane 1-sulphonate (Isomer 2).

FIG. 2: Depicts ¹³C NMR of sodium 2-hydroxy propane 1-sulphonate (Isomer 2).

### DETAILED DESCRIPTION OF THE INVENTION

Global warming caused due to excessive use of fossil fuels (while transporting or manufacturing processes) has led to increased awareness to consumers and hence there is a need of product that will significantly reduce the plastic usage and save on energy consumption ultimately leading to reduction in damage caused to environment. This has been achieved by formulating highly concentrated solid or liquid-based composition that can be diluted or reconstituted at the consumers end. Solid forms have serious limitation as it requires energy intensive processes for converting the liquid composition into solid form. Further, use of polymeric gums and thickeners leads to compromise in performance is another limitation. IN202221026146 discloses solid powder based concentrated formulation that avoids usage of polymeric gums and thickeners but teaches a process that requires spray drying, an energy intensive process. US 2021/0220243 A1 discloses a concentrated surfactant combination with one or more structuring agents to achieve the final desirable physical attributes, such as increase in viscosity upon reconstituting with water to create the end-use compositions. However, very high % of structuring agent like lauric acid particularly at higher pH zones, specially lauric acid exists as a soap that has deleterious effects on the skin and hair. The high % of structuring agent like lauric acid also results in foam depressant in the acidic zone of the pH (in formulations with pH similar to skin pH).

These shortcomings have been overcome by the concentrated compositions of the present invention by totally eliminating the structuring agent or structurant and achieving the final customary attributes of ‘sulphate-free’ personal cleansers in terms of transparency, foam and lather and gentle cleansing at skin pH. The concentrated compositions of the present invention are made with ‘sulphate-free’ anionic surfactants as the primary surfactants, zwitterionic surfactant as the secondary surfactant and preservatives that are non-toxic and one or more personal care ingredient/actives. The anionic surfactants are always in higher percentage relative to zwitterionic surfactant in the compositions of present invention.

### Anionic Surfactants:

The concentrated reconstitutable liquid composition of the present invention offers, upon dilution with, personal end-wash composition that is stable and with customary physical attributes and excellent performance and aesthetics at skin pH. This has been achieved by unexpected discovery that combination of sodium lauroyl methyl taurate (Formula 1) and sodium lauroyl methyl isethionate (Formula 2) in molar ratio of 1: 2 can be produced at 44-45% solids concentration in aqueous media by the process described in U.S. Pat. No. 9,308,156 B2 using lauroyl chloride in a pseudo one pot process. This process is affected by reacting dry powder of sodium methyl isethionate with part of the lauroyl chloride and hydrochloric gas generated is scrubbed by absorbing into water. The excess lauroyl chloride in the reaction pot (containing sodium lauroyl methyl isethionate) is then reacted with sod N-methyl taurine in water in the presence of a base to form aqueous solution sodium N-lauroyl, methyl taurate along with sodium O-lauroyl methyl isethionate. Sodium methyl isethionate is produced by reacting sodium bisulphite and propylene oxide exactly in the same analogous manner following the guidelines and procedure for sodium isethionate by reacting sodium bisulphite and ethylene oxide) sodium isethionate (DE 569148 (1931) IG Farben industrie AG). Schenk et al. reported synthesis of sodium methyl isethionate and the possibility of two isomers. (Schenck and Kaizerman, ‘Reaction of bisulphite with epoxy compounds’, Journal of American Chemical Society Vol 75 1636-1641(1953), However, later in 1970, Belgian scientists conclusively proved that sodium 2-hydroxy propane 1-sulphonate (Isomer 2) is formed exclusively and not sodium 1-hydroxy-2 sulphonate (Isomer 1) from the reaction of propylene oxide and sodium bisulphite. (J. Bombeke and E. J. Goethals; ‘The reaction of sodium bisulphite with propylene oxide’ Bull. Soc. Chim. Belges, 79, 157-159 (1970)).

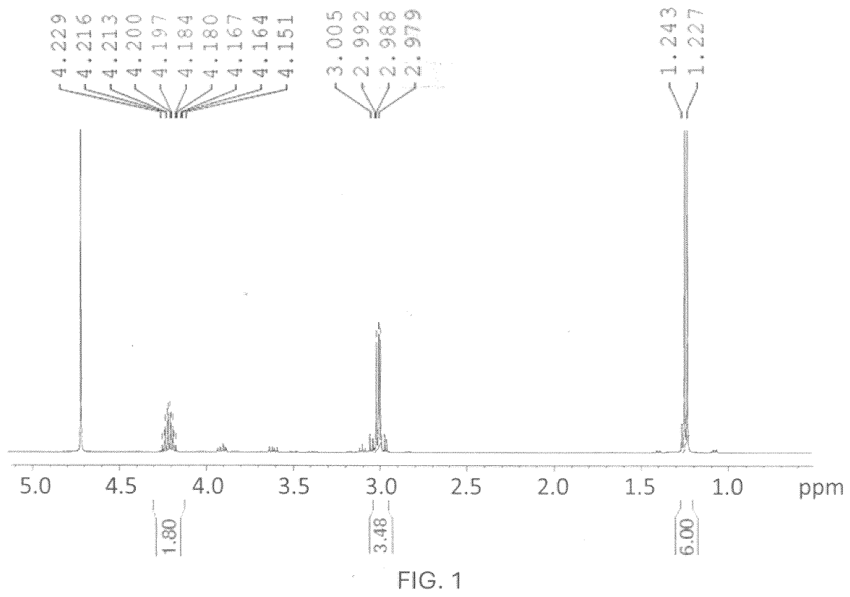

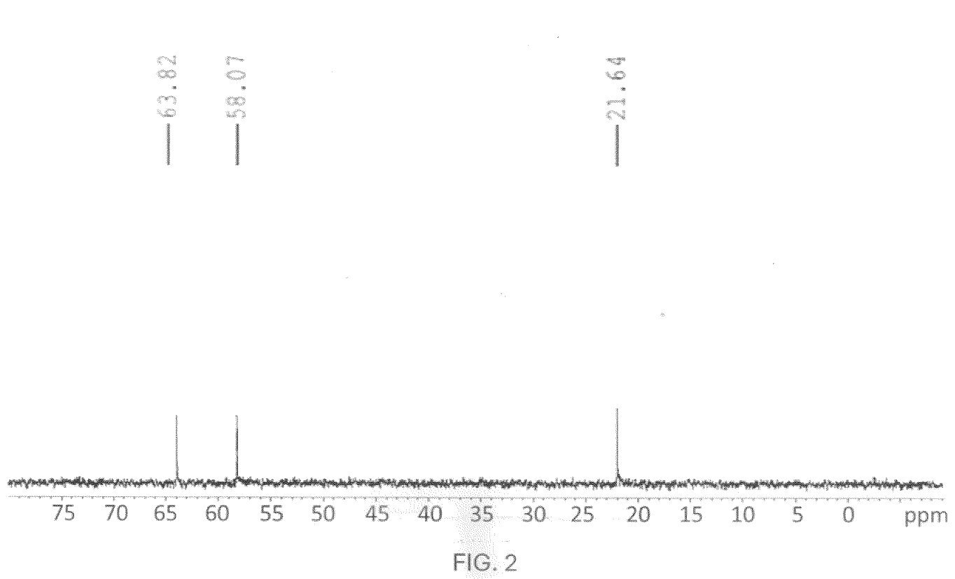

The above reported observation is confirmed by the ¹H NMR ((D₂O) analysis of the dried sample of sodium methyl isethionate (FIG. 1 and FIG. 2) that sodium 2-hydroxy propane 1-sulphonate (Isomer 2) is the predominant isomer. Methyl protons get split into a doublet (81.22 to 1.24), the most down fielded proton (on the carbon that is connected to hydroxyl oxygen) is a multiplet between 8 4.15 to 4.22. This multiplet integrates for one proton and that is split by adjacent three protons of methyl and two protons of methylene group. The methylene proton of Isomer 2 shows up at 8 2.97 to 3.0 as finely split doublet due to adjacent chiral proton. ¹³C NMR (D₂O) reveals only three signals for three carbons at 8 21.64 (methyl), 8 58.07 (methylene attached sulphur) and 8 63.82 (methine attached oxygen). Methyl carbon of isomer 1 appears at around 8 12.

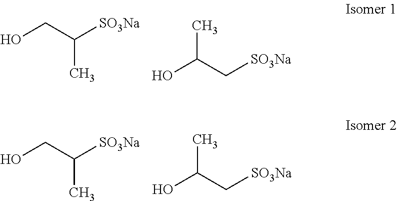

Sexton et al. reported the continuous manufacturing process (U.S. Pat. No. 2,810,747A, (1957), ‘Continuous production of salts of hydroxy aliphatic acids’). Based on the literature reports, the reaction is carried out at lower temperature for better control of exotherm by controlling the addition of propylene oxide. The details are given in Example section of present invention.

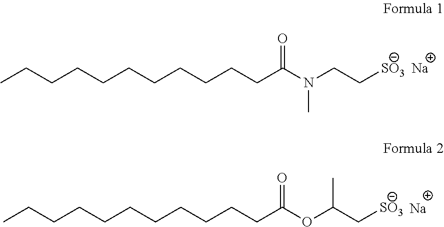

IN 202221026146 reports the combination of sodium N-acyl methyl taurate and sodium N-acyl methyl isethionate in the ratio 1:1 and 2:1 using corresponding acyl chloride. These combinations with molar ratio of 1:1 or 2:1 with sodium lauroyl methyl taurate and sodium lauroyl methyl isethionate are produced with around 30% solids content. Beyond this concentration (higher solids content), the reaction mass becomes too viscous to stir. However, unexpectedly it is discovered that if the ratio between the two mild surfactants (say, sod lauroyl methyl taurate to sod. lauroyl methyl isethionate) is reversed (that is from 2:1 to 1:2) then it is possible to produce a very high active concentrated blend of these two surfactants (i.e. sodium lauroyl methyl taurate and sodium lauroyl methyl isethionate in molar ratio of 1:2) at 44 to 45% solids content with viscosity of around 1500-3500 cps. The detailed process of making (along with the spectral and chemical analytical data) it is described in Example 1 of the present invention.

### Zwitterionic Surfactant:

Anionic surfactant combination of 45% solids level is made with the unexpected discovery of 1:2 ratio of Acyl taurate to acyl isethionate however, to make high active combination of primary anionic surfactants and secondary zwitterionic surfactants, it is necessary to have the secondary surfactant available in around same or higher activity (concentration). In this case secondary zwitterionic surfactant used for personal cleansing compositions is, cocamidopropyl betaine, that is available as 29% active with about 35 to 36% solids content. Other high active forms are usually reported and commercially available wherein some additional process aids are used. For example, U.S. Pat. No. 5,354,906 (1994) deploys free coco fatty acid (around 3%) to keep it fluid during the process and to get the final concentration at 45% solids. A decade later, (U.S. Pat. No. 7,384,898 (2008)) similar high active cocamidopropyl betaine (in aqueous solution) is reported with another additive like cocoyl glycine around 3% (N-acyl amino acids) to keep reaction mass fluid enough (mixable) to achieve the final concentration of the betaine around 45%. There are several other approaches listed in background section of U.S. Pat. No. 7,384,898. There are several processes reported for making high active cocamidopropyl betaine by conducting reaction in hydro-alcoholic solvents and thereby keeping the reaction mixture fluid at high concentration (U.S. Pat. Nos. 4,832,871, 6,335,370, 7,033,989). Interestingly, EP 1659109 discloses a process of keeping betaines fluid by generating about 10% betaines derived from lower aliphatic acids during the synthesis so that reaction mass can be agitated at higher concentration and final product of around 60% is achieved.

This challenge of making high active cocamidopropyl betaine without any additive is addressed in present invention by conducting the reaction in cocamidopropyl betaine of commercial grade (36%) as a medium. The reactants concentration is then adjusted to get the final solids content around 44-45% level. The initial quantity of CAPB (36% solids) taken is about 15% of the total batch size. Coco fatty acid used to make the cocoamidopropyl dimethyl amine has alkyl chain distribution of C12 to C18 hydrogenated with a molecular weight of 218. The details of alkyl chain distribution and the analysis of intermediate cocoamidopropyldimethyl amine are given in the Experimental Section. The compositions described in this invention are explained with cocamidopropyl betaine as the zwitterionic surfactant however, with other zwitterionic surfactants like lauramidopropyl betaine or cocoamidopropylhydroxy sultaine or lauramidopropyl hydroxy sultaine can be used in place of cocoamidopropylbetaine.

### Antimicrobial Preservatives:

In terms of antimicrobial preservatives, all approved and listed antimicrobials can be used. The base formula contains benzoic acid and that can be complimented by other non-controversial and relatively non-toxic antimicrobials selected from sorbic acid, dehydroacetic acid, capryloyl glycine, undecylenoyl glycine and phenoxy ethanol. Some other fermentation derived diols like 1,3 propane diol or any other 1,2 hexane diol or 1,2 caprylyl glycol.

The antimicrobial preservative is present in 1.0% to 2.5% by weight of total composition.

### Cosmetic and Pharmaceutical Auxiliaries:

A personal care composition can include variety of cosmetically acceptable additives and actives. This includes a range of actives, conditioning agent, sunscreen, prebiotics and postbiotics, carbohydrate polymer, etc. Suitable examples of these auxiliary agents added in the concentrated composition of present invention and the viscosities of concentrate and reconstituted composition are provided in table 5 of Example 1.

Such cosmetically or pharmaceutically acceptable auxiliaries are selected from one or more hair care actives, skin care actives, water-soluble dry herbal extracts, vitamins, chelating agents, inorganic salts, moisturizing agents, soothing agents, nourishing agents, conditioners, colors and fragrances.

The cosmetically and pharmaceutically acceptable auxiliaries are used in 0.5% to 5% by weight of total composition.

The present invention provides a process which is specifically designed to render the blend of anionic surfactants in a high concentration and final concentrate having minimum 45% solid without the use of any structurant.

Blending of high active anionic surfactant mix (step 1 and 2 of Example 1), cocamidopropyl betaine, preservative (benzoic acid) is done at room temperature to create the ‘base composition’ (Example 1). This base surfactant composition, in general, is prepared wherein the anionic surfactants are blended with zwitterionic surfactant in the weight ratio 5.0 to 6.0:3.5 to 4.5. The pH is adjusted to 5.5-6.0 if necessary. The composition with 44-45% solids content exhibits viscosity of around 2000 cps at room temperature. This composition of Example 1 on dilution with water 3 times and 4 times results in stable compositions with viscosities of 7000 and 4000 and pH of around 5.5 to 5.8. Therefore, in an embodiment the said composition is diluted 3-5 times. The reconstituted compositions with around 10 to 15% solids content can have customary consumer desired attributes of viscosity and the pH. These compositions exhibit good gentle cleansing performance with the desired degree of foam and lather at a pH that is close to natural pH of the skin. The concentrated reconstitutable composition of Example 1 (base composition) is without any personal care actives. Its solids content is 45% and pH of 5.5 and has viscosity of 1500 cps (Table 1). The viscosity of 1500 cps for the concentrate form gives scope for increasing the solids content further with some increase in the viscosity.

The table 4 in Example 1 indicates that 25 grams of liquid of 45% solids content is mixed with 75 g of water gives 100 g of liquid composition with solids of 11.25%. This means that original solids content of 45% is diluted four times giving the final solids content of 11.25%. Viscosity after 4 times dilution is 7450 cps and pH of 5.6. Similarly, three times dilution of the base material afforded viscosity of 8600 cps with pH of 5.5. This base surfactant composition of Example 1, wherein anionic surfactants (sodium lauroyl methyl taurate and sodium lauroyl methyl isethionate) are blended with zwitterionic surfactant, cocamidopropyl betaine) in the weight ratio 5.0 to 6.0: 3.5 to 4.5, is tested with a variety of personal care ingredients for their impact on the rheological properties both at the ‘concentrated’ precursor stage and at the ‘diluted or reconstituted’ stage. The personal care ingredients can be either liquids or solids. These can be either water-soluble/water-dispersible or oil-soluble. This study is done with one ingredient at a time to determine the impact on the rheological properties of the surfactant blend in both concentrated form and after reconstituting the concentrated form with water and studying the impact at room temperature as well as at 45° C. The data is compiled in Table below (Table 5). The popular hair conditioners, particularly like behenyl trimethyl ammonium chloride (entry no 4 in the table 5) is used at 2% level and the original and the reconstituted compositions are translucent with viscosity 3000 cps and after four times dilution the viscosity builds to 10,000 cps. Similar quaternized UV absorber with a methoxycinnamido chromophore and a long behenyl chain (entry no 5 of table 5) had small drop in the viscosity in concentrated form as compared to the base formula of Example 1 and upon dilution four time with water, viscosity of 7200 cps is achieved. With methoxy cinnamidopropyl dimethyl hydroxy sultaine, a water-soluble UV absorber, with same UV absorbing moiety and sulphobetaine type of structure, the rheology of the reconstituted composition does not change much (entry no 12). The other hair conditioners like stearamidopropyl dimethyl amine (entry no 1, Table), and Polyquaternium 10 (entry no 3) increase viscosity of the base blend at 2.0% to around 8000 cps and upon dilution by four times with water the viscosity is maintained at 8000 cps. Interestingly, behenamidopropyl dimethyl amine (entry no 2) shows a perfect behaviour of affording a low viscous concentrate viscosity of 1820 cps) and high viscous (6540 cps) after reconstituting by four times dilution. As regards silicones, BELSIL ADM 22, an amodimethicone (entry 6) is compatible with the base composition of Example 1. At 2% level of incorporation, it gives transparent composition after dilution (2490 cps) which is higher than the viscosity of original concentrated composition (1800 cps). Another polydimethylsiloxane in anionic emulsion form (Xiameter-MEM 1785) at 2.0% in the concentrate of Example 1 gives satisfactory viscosity results after reconstituting with water (entry no 7). Similar behaviour is observed with another carbohydrate based cationic polymer, PQ 10 where there is no drop in the viscosity after reconstituting the concentrate with the cellulosic quat is present at 2% (entry no 3). Another carbohydrate based popular personal care ingredient is hydroxypropyl starch phosphate (entry no 9) which shows very desired behaviour exhibiting lower viscosity (2564 cps) for the precursor concentrate and higher viscosity (8300 cps) after reconstituting (dilution) with water four times. Similar behaviour is exhibited by 12-hydroxy stearic acid at 2% level (entry no 10) wherein the diluted product is opaque. The bulky molecules seem to be favouring this phenomenon as exhibited by the perfect behaviour by Vit E acetate (entry no 16) and sodium stearoyl lactylate (entry no 13). Surprisingly, a totally hydrophilic small molecule like glycerin (entry no 11) is found to be ideally suited for this concept of reconstituting the precursor to get significantly higher viscosity. Other gentle and totally green surfactants like sophorolipids made from oleic acid and glucose, entry no 17) and alkyl polyglucosides (entry no 18 and 19) support the expected performance. Entry no 8 shows the utility of commonly used pearlizer, ethylene glycol distearate in cold dispersible form is quite suitable for creating pearly end-compositions. Though the base composition has benzoic acid at 2% level, there would be a need for the additional antimicrobials after the dilution because depending upon the extent of dilution, the amount of water goes up with a simultaneous decrease in the solids content. Hence a few compositions of gentle and eco-friendly antimicrobials (entry no 14, and 15) are examined for their suitability Thus, it can be seen from the Table in example section that a variety of ingredients are tolerated by the unique combination of anionic and zwitterionic surfactants with respect to the stable rheology behaviour after the dilution with water. All the diluted (reconstituted) compositions are stable with respect to pH and the viscosity.

Using some of the personal care ingredients including polymeric conditioners, mild preservatives and fragrances, a few concentrated compositions (hair cleansers and body cleansers) are made and studied after reconstituting (Examples 2 to 9) them with water. The shampoo concentrates made with a variety of conditioners (silicones and quaternaries including the carbohydrate types) and with water-soluble and water-insoluble fragrances are in the zone of 1000 to 3000 cps viscosities. On dilution four times with water, the viscosities are in the zone of 2000-10000 cps that is quite customary for rinse-off hair cleansing formulations. Example 2 is shampoo concentrate with amodimethicone and quaternary ammonium conditioner and a quaternary hair color protector and preservative system of gentle antimicrobials. The viscosity of this concentrate at 46% is less than thousand cps and four times dilution builds it to viscosity of 1500 cps and with pH of 5.5. The conditioning shampoos of Example 2 and 3 deploy oil-soluble fragrance and preservation systems that are slightly different from each other. The reconstituted composition of Example 3 is tested for its antimicrobial efficacy and passes the challenge test that is performed as per the protocol prescribed by PCPC. Conditioning shampoo of Example 4 is similar to composition of Example 2 with exception of water-soluble fragrance that resulted in significant jump in the viscosity (6000 cps) of reconstituted composition. Example 5 is pearly shampoo concentrate with water soluble fragrance and ethylene glycol distearate, the pearlizer, in cold-dispersible form, quaternized UV absorber and conditioner and gentle antimicrobials for preservation, affords viscosity of 1220 cps at 46% solids. This concentrate of Example 5, on reconstituting with water gives excellent viscosity of 5800 cps at skin pH of 5.5 which is very customary to ‘sulphate-free’ shampoos.

Similarly, bodywash concentrates are made with solids contents of upwards of 45% and with viscosities around 2000 cps with fragrances and preservatives. Upon four times dilution the body wash concentrates afford compositions with customary viscosities. (2000-10000 cps).

Example 6 discloses a body wash concentrate with an emollient, sodium stearoyl lactylate. It shows very moderate viscosity of 4000 cps after dilution with water (four times) at pH of 5.5. The microbial robustness is tested by the PCPC ‘challenge test’ protocol and it passes the test comfortably. The concentrated pearly body wash composition of Example 7 has slightly higher solids content and dilution it affords impressive viscosity of 8000 cps. Examples 8 and 9 are designed with green (alkyl polyglucoside) and natural surfactants (sophorolipids) to create transparent and pearly bodywashes that are expected to be ultra-mild towards the constituents of the skin, namely, the lipids and the proteins.

All compositions described in Examples 2 to 9 (the concentrated precursors as well as the reconstituted end-use compositions) have cleared the stability tests at ambient temperature as well as at 45° C. for a month. The reconstituted compositions of Example 3 and Example 6 are subjected to ‘challenge test’ (preservation efficacy) as per the PCPC protocol and both compositions cleared the ‘challenge tests’ indicating that they are adequately robust against any accidental microbial contamination.

### Advantages:

1) The free-flowing, hydratable, reconstitutable and concentrated liquid compositions of the present invention are useful for saving significant amount of plastic that is used in packaging the personal cleanser formulations such as shampoos and bodywashes. The concentrated from saves significant amount of water and significant saving on energy spent during processing and during transportation contribution to water conservation as well as less generation of greenhouse gases.

2) The compositions of this patent application overcome the drawbacks of the prior art. The free-flowing, hydratable, reconstitutable and concentrated liquid compositions of the present invention are based on only surfactants and personal care actives. These compositions do not depend upon any rheology modifier (polymeric viscosity booster) or a structuring agent (C6-C14 acids and alcohols, US 2021/0220243 A1) for achieving customary viscous appearance and aesthetics of the cleansing end-compositions. The limitations imposed by the use of structuring agents like lauric acid are described in the background section.

3) The compositions of this patent application overcome the drawback of the prior art which is based on dry format made by energy-intensive drying of surfactants. The dry format of concentrated compositions puts serious limitations on use of personal care actives that are liquid in nature. The free-flowing, hydratable, reconstitutable and concentrated liquid compositions of the present invention accommodate all sorts of personal ingredients regardless of their nature of either solid or liquid (Table 5). This is a clear advantage over the powder composition which can't accommodate liquid ingredients, thus imposing serious limitation on the performance of the end-formulation (Indian Patent appl IN 202221026146 by Koshti et al.)

4) A number of skin and hair actives (Table 5) are added at room temperature (cold processing) to the free-flowing, hydratable and concentrated liquid compositions of present the invention resulting in significant energy saving.

5) Unlike the prior art where energy is spent in drying the aqueous solutions of surfactants to get them in solid form (Indian Patent appl IN 202221026146 by Koshti et al.), the concentrated, hydratable liquid compositions of the present invention are energy efficient for two reasons a) drying of aqueous solutions is avoided and b) these compositions accommodate personal care actives without expending any energy for heating whole mass of the concentrated composition.

6) The free-flowing, hydratable, reconstitutable and concentrated liquid compositions of the present invention are very easy to use for the concept of ‘do-it-yourself’ wherein consumer is directed to dilute it with water and shake the refill container gently.

7) Upon dilution with water, the compositions of the present invention result into transparent compositions with a pH close to natural pH of skin with customary appearance (viscous nature).

8) The concentrated hydratable and reconstitutable compositions result into personal care cleansing compositions that are made from ‘sulphate-free’ mild surfactants and offer consumer desired in-use performance in terms of superlative foam and lather at skin pH.

### EXAMPLES

The present invention is now described by way of non-limiting illustrative examples. The details of the invention provided in the following examples are given by the way of illustration only and should not be construed to limit the scope of the present invention.

The common personal ingredients (CAS numbers are given in Table 5) are procured from generic sources. The formulated products are identified by their trade-names and are sourced from the manufacturers or their distributers. Silicones are sourced from Dow (Xiameter) and Wacker Chemie (BELSIL). Antimicrobials combinations (Galguards) are procured from Galaxy. Quaternized UV absorber cum conditioner, GalHueShield, and the water-soluble UV-absorber, Galaxy SunBeat, the cold-peralizer Sparkle 670, are procured from Galaxy Surfactants Ltd, India. Some surface-active emollients like sodium lauroyl lactylate, sodium stearoyl lactylate, zwitterionic surfactant, cocamidopropyl betaine are sourced from Galaxy Surfactants Ltd, India. Hydroxy propyl starch phosphate (Structure XL) is purchased from Nouryon, Amsterdam, Netherlands. The fragrances are procured from Imperial Fragrances and Flavours, Howrah, India.

### Example 1

Preparation of low viscous, hydratable surfactant concentrate that thickens upon dilution with water:

The synthesis involves four steps: 1) preparation of sodium methyl isethionate in dry form, 2) preparation of blend of anionic surfactants, sodium methyl lauroyl isethionate and sodium methyl lauroyl taurate using lauroyl chloride and with minimum solids content of 40%, 3) preparation of zwitterionic surfactant, cocamidopropyl betaine, with minimum solids content of 40%, and 4) blending of the anionic surfactant composition of step 2 and zwitterionic surfactant of step 3.

### Step 1:

### Sodium Methyl Isethionate:

Aqueous solution of sodium bisulphite is reacted with propylene oxide as per the literature procedure described in (DE 569148 (1931) IG Farben industrie AG). The literature reference mentioned below are also referred to.

[(Schenck and Kaizerman, ‘Reaction of bisulphite with epoxy compounds’ in Journal of American Chemical Society, Vol 75 1636-1641(1953), U.S. Pat. No. 2,810,747A (1957), and Sexton et. al. ‘Continuous production of salts of hydroxy aliphatic acids’ (U.S. Pat. No. 2,810,747A, (1957))]

It is then spray-dried to convert in powder form with typical active matter of 95%, and moisture content of 5% maximum and traces of propylene glycol and sodium bisulphite.

In a five-litre pressure vessel, aqueous solution of sodium metabisulphite is charged (40%, 520 ML (300 g, 1.57 mmol of metabisulphite which is equivalent of 3.4 mmol of sod bisulphite,) and pH is adjusted between 6.5 to 7.0 with sodium hydroxide. The air inside the pressure vessel is then removed by several cycles of nitrogen and vacuum. The vessel is pressured with nitrogen up to 1 kg/m2. The solution of metabisulphite is then heated to 70° C. and propylene oxide (138 g, 3.1 gmol) is then slowly introduced to the stirred sodium bisulphite solution under nitrogen while controlling the exotherm (circulating cold water through the cooling coil) and maintain the temperature between 70 to 80° C. The completion of the reaction is indicated by the pressure drop to original nitrogen pressure. The aqueous solution of sodium methyl isethionate has the following analysis: practically colorless (APHA 15 units, solids content 45%, and propylene glycol 1%. The reaction mass is cooled and then spray-dried to get a fine powder of sodium methyl isethionate with active matter of 98%, moisture content of 1.5% and traces of propylene glycol and sodium bisulphite. This reaction is expected to give sodium 2-hydroxy propane 1-sulphonate exclusively as per Bombeke et al. (J. Bombeke and E. J. Goethals; ‘The reaction of sodium bisulphite with propylene oxide’ Bull Soc. Chim. Belges, 79, 157-159 (1970))

This is confirmed by recording the NMRs both on ¹H and ¹³C NMR on Brucker machine, 400 MHz). Chemical shifts and interpretation are discussed in ‘Detailed Discussion’.

### Step 2:

### Synthesis of the Blend of Sodium Lauroyl Methyl Taurate and Sodium Lauroyl Methyl Isethionate (1:2):

The synthesis of this blend (44% solids content) of sodium N-lauroyl methyl taurate and sodium O-lauroyl methyl isethionate is synthesized as per the procedure described in U.S. Pat. No. 9,308,156 by Koshti et al. Fatty acid (alkyl chain distribution C8: 0.5%, C10: 0.5%, C12: 99%) is reacted with thionyl chloride to afford lauroyl chloride as described by the ‘green’ process in U.S. Pat. No. 9,187,407.

To stirred lauroyl chloride (267 g, 1.2 gmol), under slow purging of nitrogen at room temperature, sodium methyl isethionate in powder form (133 g, 0.8 gmol) is added, and the slurry is stirred at 55-60° C. for 4 h. The HCl gas generated is absorbed in alkali solution and the progress of reaction is monitored by IR spectrum analysis. The FTIR spectrum of the intermediate shows the presence of unreacted lauroyl chloride (carbonyl stretch at 1800 cm-1), sodium lauroyl methyl isethionate (carbonyl of ester at 1734 cm-1) and disappearance of hydroxyl stretch (3323 cm-1) of sodium methyl isethionate (we can add IR image).

This fluid viscous reaction mass (365 g) is cooled to 45-55° C. and then added slowly, to a stirred aqueous, solution of sodium N-methyl taurate (170 g, 0.42 gmol) in water (425 g) along with sodium hydroxide solution (48.8%, 67 g, 0.79 gmol) simultaneously while maintaining the pH of the reaction mass between 10.5 to 11.5 and the temperature between 20 to 40° C. The addition is completed in two hours and the reaction mass is stirred for another 4 h at 25-30° C. The pH is adjusted to 7.5 with 2% benzoic acid and HCl, if necessary. The solids content of the reaction mass is adjusted to 44% to yield 1025 g of aqueous solution.

IR spectrum of dried sample shows carbonyl stretch of amide at 1600-1620 cm-1 and NH stretch at 3344 cm-1. The other significant stretching frequencies are carbonyl of ester of alkanoyl isethionate at 1734 cm-1 and total disappearance of carbonyl stretching frequency (1800 cm-1) of lauroyl chloride.

### Step 3:

### Synthesis of Concentrated Cocoamidopropyl Betaine

Cocofatty acid: Alkyl chain distribution C12: 56.84%, C14: 23.69%, C16: 9.10, C18: 10.07%)

Cocofatty acid ((218 g, 1.0 mol) is heated with N, N-dimethylpropyl diamine (105 g 1.03 mol) under nitrogen blanket at 165-170° C. for 12 hours. Unreacted diamine 0.2%, unreacted fatty acid 2.0%.

To a stirred aqueous solution of cocamidopropyl betaine (110 g, 36% solids), under nitrogen blanket at 85° C., cocamidopropyl dimethyl amine (201 g, 0.6 gmol) and sodium monochloro acetate (210 g, 35% solution, 0.63 gmol) are added simultaneously over a period of 1.0 h and stirring is continued at 85° C. for 5 hrs. The progress of reaction is monitored by estimating the liberated chloride ions and pH is maintained at 8.0-8.5 throughout the reaction. After quantitative liberation of chloride ions, the reaction is stopped to afford cocamidopropyl betaine (676 g) as pale yellow colored liquid with the following analysis.

Solids content: 43.35%, sodium chloride content: 6.35%, pH (as such): 5.10 and unreacted cocamidopropyl dimethyl amine: 0.45%.

### Step 4:

Blending of products of Step 2 (blend of sodium lauroyl methyl taurate and sodium lauroyl methyl isethionate in the molar ratio of 1:2 with overall solids content of 44% with the product of step 3 (cocamidopropyl betaine with solids content of 44%)

Products of Step 2 and Step 3 are blended together at room temperature until homogeneous and to it benzoic acid (1.5 g), is added and if necessary, pH is adjusted to 5.5.

TABLE 1
Concentrated reconstitutable composition as per present invention:

| Concentrated composition | Grams |
|---|---|
| Product of Step 2 (sodium lauroyl methyl | 58.0 |
| taurate and sodium lauroyl methyl | |
| isethionate) | |
| Product of Step 3 (cocamidopropyl | 40.0 |
| betaine) | |
| Benzoic acid | 1.5 |
| Analysis of product of step 4 | |
| Appearance | Low viscous translucent liquid |
| %, solids | 45 |
| pH (as such) | 5.5 |
| Viscosity | 1500 cps |
| Sodium chloride | 6.0% |

### Comparative Examples

The present invention provides a process which is specifically designed to render the blend of anionic surfactants in a high concentration and final concentrate having minimum 45% solid without the use of any structurant.

The inventors of the present invention discovered a specific ratio of the two anionic surfactants (sodium lauroyl methyl taurate and sodium lauroyl methyl isethionate; in 1:2) which enables to produce the blend in minimum 40% solid concentration. Beyond this ratio achieving the blend of the two anionic surfactants in such a high solid content with desired viscosity is not possible (Table 2).

TABLE 2
Comparative examples (CE) with varying ratio of sodium
lauroyl methyl taurate and sodium lauroyl methyl
isethionate (CE1 1:1, CE2 2:1 and CE3 3:1)

| | | Comparative | Comparative | Comparative |
|---|---|---|---|---|
| Sr. | | example 1 | example 2 | example 3 |
| No. | Parameters | (1:1) | (2:1) | (3:1) |
| 1 | Maximum solid | 32% | 34% | 37.5% |
| | content | | | |
| 2 | Appearance | Clear Viscous | Clear Viscous | Clear |
| | | Liquid | Liquid | Viscous |
| | | | | Liquid |
| 3 | pH (as such) | 7.45 | 7.45 | 7.55 |
| 4 | viscosity | 3800 cp | 3200 cp | 1250 cp |
| 5 | NaCl | 4.50 | 4.65 | 5.10 |

Further, achieving the blend of sodium lauroyl methyl taurate and sodium lauroyl methyl isethionate in the ratio of 1:3 is non amenable. The addition of solid sodium methyl isethionate into lauroyl chloride makes the reaction mass very thick and non-convenient for further transfer. This results in formation of thick slurry with undesired properties.

In the other embodiment, the inventors of the present invention discovered a specific ratio of the two different types of surfactants (anionic surfactants and zwitterionic surfactants) which enables to produce the blend in minimum 40% solid concentration with desired subsequent properties.

Beyond this ratio achieving the blend of the two types of surfactants in such a high solid content with desired variation in viscosity, required ease of handling is not possible (Table 3).

TABLE 3
Comparative examples (CE) with varying ratio of blend
of anionic surfactants and zwitterionic surfactant

| | Comparative | Comparative | Comparative |
|---|---|---|---|
| Description | example 4 | example 5 | example 6 |
| Ratio of a:b | 1:1.5 | 4:1 | 1:4 |
| % Solids | ~46% | ~46% | ~46% |
| Appearance | Non flowable | Clear low | white lumpy |
| | Clear viscous | viscous liquid | solid |
| | liquid | | |
| pH | Non- | 5.59 | Non- |
| | measurable | | measurable |
| Viscosity of | Non- | 1250 | Non- |
| concentrate | measurable | | measurable |
| Viscosity | Non-dilutable | 210 | Non-dilutable |
| after dilution | | | |

The deviation from the claimed ratio of anionic surfactants blend to zwitterionic surfactant results in the compositions deviating in the required specification. Reversing the ratio of blend of anionic surfactants and zwitterionic surfactant as 1: 1.5 produces non flowable clear viscous liquid which solidifies on cooling (Comparative example 4). A composition made with the blend of anionic surfactants and zwitterionic surfactant in 4:1 ratio fails to achieve desired viscosity on dilution. Rather a miserable drop in viscosity is observed (Comparative example 5). A composition made with blend of anionic surfactants and zwitterionic surfactant in 1:4 ratio fails the initial specification and renders thick white solid instead of flowable liquid (Comparative example 6).

### Dilution of Blend of Example 1 with Water

The above concentrate is diluted three times and four times by gentle stirring with a glass rod or by shaking in bottle with stopper for a couple of minutes. The analysis performed after settling of foam is given in the table below.

TABLE 4
Analysis of diluted concentrate of table 1

| | | |
|---|---|---|
| Dilution | Three times | Four times |

| | | | | |
|---|---|---|---|---|
| Weight of | 30 | g | 25 | g |
| Concentrate | | | | |
| Weight of Water | 70 | g | 75 | g |

| | | |
|---|---|---|
| Nature | Transparent Viscous | Transparent Viscous |
| | Liquid | Liquid |
| % NaCl | 2.0% | 1.5% |
| pH (as such) | 5.5 | 5.6 |

| | | | | |
|---|---|---|---|---|
| Viscosity (25 dec | 8600 | cp | 7450 | cp |
| C) | | | | |

TABLE 5
Concentrated formulation of present invention with different
actives and their viscosity before and after dilution

| | | | | Viscosity | |
|---|---|---|---|---|---|
| | | % | | after four | |
| Sr | | Added | Viscosity | times | |
| No | Ingredients and CAS Nos | active | original | dilution | Appearance |
| | Blend of Example 1 | | 1550 | 8250 | Clear |
| | Sodium lauroyl methyl | | | | |
| | isethionate (928663-45-0), | | | | |
| | Sodium lauroyl methyl | | | | |
| | taurate (4337-75-1) | | | | |
| | Cocamidopropyl | | | | |
| | betaine (61789-40-0) | | | | |
| | benzoic acid (65-85-0) | | | | |
| 1 | Stearamidopropyl dimethyl | 2 | 8000 | 7990 | Clear |
| | amine (7651-02-7) | | | | |
| 2 | Behenamidopropyl | 2 | 1820 | 6540 | Clear |
| | dimethylamine | | | | |
| | (60270-33-9) | | | | |
| 3 | Hydroxyethyl trimethyl | 2 | 7425 | 8130 | Clear |
| | ammo chloride cellulose | | | | |
| | (PQ-10) | | | | |
| | (67-48-1) | | | | |
| 4 | Behenyl trimethyl ammo | 2 | 3000 | 10,000 | Clear |
| | chloride (4292-25-5) | | | | |
| 5 | GalHueShield | 2 | 950 | 7200 | Clear |
| | Methoxy cinnamidopropyl | | | | |
| | behenyl dimethyl ammo. | | | | |
| | chloride (880645-41-0) | | | | |
| 6 | BELSIL ADM 22 | 2 | 1800 | 2490 | Clear |
| | Amodimethionce, | | | | |
| | (99363-37-8) | | | | |
| | Laureth 7, (68439-50-9) | | | | |
| | Trideceth 3 (69011-36-5) | | | | |
| | Laureth 6 (9043-30-5) | | | | |
| 7 | Xiameter-MEM 1785 | 2 | 2000 | 2550 | Opaque |
| | Benzenesulfonic acid, | | | | |
| | C10-16-alkyl derivs., | | | | |
| | compds. with | | | | |
| | triethanolamine | | | | |
| | (68584-25-8) | | | | |
| | Octamethyl | | | | |
| | Cyclotetrasiloxane | | | | |
| | (556-67-2) | | | | |
| 8 | Galaxy Sparkle 670 | 3 | 1020 | 9850 | Pearly |
| | Glycol Distearate | | | | |
| | (91031-31-1), CAPB | | | | |
| | (61789-40-0), Laureth 4 | | | | |
| | (68439-50-9) | | | | |
| 9 | Hydroxypropyl starch | 2 | 2564 | 8300 | Clear |
| | phosphate (Structure XL) | | | | |
| | (113894-92-1) | | | | |
| 10 | 12 hydroxy stearic acid | 2 | 2430 | 9460 | Opaque |
| | (106-14-9) | | | | |
| 11 | Glycerin (56-81-5) | 3 | 570 | 9865 | Clear |
| 12 | Galaxy SunBeat | 2 | 2078 | 2530 | Clear |
| | Methoxy cinnamidopropyl | | | | |
| | dimethyl hydroxy sultaine | | | | |
| | (500731-87-3) | | | | |
| 13 | Sodium stearoyl lactylate | 2 | 630 | 9750 | Clear |
| | (25383-99-7) | | | | |
| 14 | Galguard Trident S | 2 | 1520 | 3550 | Clear |
| | Benzoic acid (65-85-0), | | | | |
| | Capryloyl glycine | | | | |
| | (14246-53-8), Undecylenoyl | | | | |
| | glycine (54301-26-7). | | | | |
| 15 | Galguard Tetra | 2.25 | 620 | 3680 | Clear |
| | Benzoic acid (65-85-0), | | | | |
| | Capryloyl glycine | | | | |
| | (14246-53-8), Undecylenoyl | | | | |
| | glycine (54301-26-7), | | | | |
| | Phenoxyethanol (122-99-6) | | | | |
| 16 | Vit E acetate | 0.5 | 920 | 9800 | Clear |
| | 7695-91-2 | | | | |
| 17 | Sophorolipids | 2 | 380 | 5600 | Clear |
| | (Galsophorin) | | | | |
| | 2102536-64-9 | | | | |
| 18 | Decyl glucoside | 2 | 740 | 4620 | Clear |
| | 68515-73-1 | | | | |
| 19 | Lauryl glucoside | 2 | 2120 | 5070 | Clear |

### Typical Process for Making Cleansing Formulations Using the Blend of Example 1

All ingredients taken in following examples are cold processable, including the pearlizer or silicones or preservatives. All are mixed together till the mass becomes homogeneous. The viscosities of the concentrates are in the zone of 500 to 4000 cps and 2000 to 10000 cps on dilution four times with water.

### Example 2: Conditioning Shampoo Concentrate and the Reconstituted Shampoo

| Ingredients | Weight |
|---|---|
| Blend of Example 1 | 92 |
| BELSIL ADM 22 | 1 |
| Behenyl trimethyl ammonium chloride | 2 |
| Methoxy cinnamidopropyldimehyl | 1 |
| behenyl ammonium chloride | |
| Galguard Trident S (benzoic acid, capryloyl | 1 |
| glycine and undecylenoyl glycine) | |
| Water Lily- 12366 | 1 |
| Analysis of concentrate | |
| Appearance | Translucent |
| pH | 5.8 |
| Viscosity cps | 900 |
| Solids | 46% |
| Analysis after Four Times dilution | |
| Appearance | Clear |
| pH | 5.45 |
| Viscosity cps | 1500 cps |

### Example 3: Conditioning Shampoo Concentrate and the Reconstituted Shampoo

| Ingredients | Weight |
|---|---|
| Blend of Example 1 | 92 |
| BELSIL ADM 22 | 1 |
| Behenyl trimethyl ammonium chloride | 2 |
| Methoxy cinnamidopropyldimethyl behenyl | 1 |
| ammonium chloride | |
| Galguard Tetra (Benzoic acid, Capryloyl | 3.0 |
| glycine, Undecylenoyl glycine & | |
| Phenoxyethanol) | |
| Strawberry & Orange -23870 | 1 |
| Analysis of concentrate | |
| Appearance | Translucent |
| pH | 5.8 |
| Viscosity cps | 980 |
| Solids | 45% |
| Analysis after Four Times dilution | |
| Appearance | Clear |
| pH | 5.45 |
| Viscosity cps | 2220 Cps |

### Example 4: Conditioning Shampoo Concentrate and the Reconstituted Shampoo

| Ingredients | Weight |
|---|---|
| Blend of Example 1 | 92 |
| BELSIL ADM 22 | 1 |
| Behenyl trimethyl ammo chloride | 2 |
| Methoxy cinnamidopropyldimehyl behenyl | 1 |
| ammonium chloride | |
| Galguard Trident S (benzoic acid, capryloyl | 1 |
| glycine and undecylenoyl glycine) | |
| Strawberry & Orange -23870 | 1 |
| Analysis of concentrate | |
| Appearance | Translucent |
| pH | 5.8 |
| Viscosity cps | 1800 |
| Solids content | 46 |
| Analysis after four times dilution | |
| Appearance | Transparent |
| pH | 5.5 |
| Viscosity cps at 25° C. | 6000 |

### Example 5: Conditioning Pearly Shampoo Concentrate and the Reconstituted Shampoo

| Ingredients | Weight |
|---|---|
| Blend of Example 1 | 90 |
| BELSIL ADM 22 | 1 |
| Behenyl trimethyl ammo chloride | 2 |
| Methoxy cinnamidopropyldimehyl behenyl | 1 |
| ammonium chloride | |
| Sparkle 670 (Ethylene glycol distearate, | 4 |
| cocoamidopropyl betaine) | |
| Galguard Trident S (benzoic acid, capryloyl | 1 |
| glycine and undecylenoyl glycine) | |
| Strawberry & Orange -23870 (water-soluble | 1 |
| fragrance) | |
| Analysis of concentrate | |
| Appearance | Pearly |
| pH | 5.8 |
| Viscosity cps | 1220 |
| Solids content % | 46 |
| Analysis after four times dilution | |
| Appearance | Pearly |
| pH | 5.5 |
| Viscosity cps at 25° C. | 5800 |

### Example 6: Bodywash Concentrate and Reconstituted Bodywash

| Ingredients | Weight |
|---|---|
| Blend of Example 1 | 93 |
| Hydroxypropyl starch phosphate | 2 |
| Sodium stearoyl lactylate | 1 |
| Galguard Tetra (Benzoic acid, Capryloyl | 3 |
| glycine, Undecylenoyl glycine & Phenoxyethanol) | |
| Water Lily- 12366 | 1 |
| Analysis of concentrate | |
| Appearance | Clear |
| pH | 5.85 |
| Viscosity cps | 1215. |
| Solids content % | 46 |
| Analysis after four times dilution | |
| Appearance | Transparent |
| pH | 5.5 |
| Viscosity cps at 25° C. | 3770 |

### Example 7: Pearly Bodywash Concentrate and Reconstituted Bodywash

| Ingredients | Weight |
|---|---|
| Blend of Example 1 | 90 |
| Hydroxypropyl starch phosphate | 2 |
| Sodium stearoyl lactyalate | 1 |
| Sparkle 670 (Ethylene glycol distearate, | 5 |
| cocoamidopropyl betaine) | |
| Galguard Trident S (Benzoic acid, capryloyl | 1 |
| glycine and undecylenoyl glycine) | |
| Water Lily- 12366 (oil-soluble fragrance) | 1 |
| Analysis of concentrate | |
| Appearance | Pearly |
| pH | 5.8 |
| Viscosity cps | 2200 |
| Solids content % | 47 |
| Analysis after four times dilution | |
| Appearance | Pearly |
| pH | 5.5 |
| Viscosity cps at 25° C. | 8000 |

### Example 8: Bodywash Concentrate and Reconstituted Bodywash

| Ingredients | Weight |
|---|---|
| Blend of Example 1 | 92 |
| Decyl glucoside | 2 |
| Glycerin | 2 |
| Sophorolipids (oleic acid and dextrose) | 2 |
| Galguard Trident S (Benzoic acid, capryloyl | 1 |
| glycine and undecylenoyl glycine) | |
| Water Lily- 12366 (oil-soluble fragrance) | 1 |
| Analysis of concentrate | |
| Appearance | Clear |
| pH | 5.8 |
| Viscosity cps | 820 cps |
| Solids content % | 46.02 |
| Analysis after four times dilution | |
| Appearance | Transparent |
| pH | 5.5 |
| Viscosity cps at 25° C. | 2000 cps |

### Example 9: Bodywash Concentrate and Reconstituted Bodywash

| Ingredients | Weight |
|---|---|
| Blend of Example 1 | 87 |
| Decyl glucoside | 2 |
| Glycerin | 2 |
| Sophorolipids (oleic acid and dextrose) | 2 |
| Sparkle 670 (Ethylene glycol distearate, | 5 |
| cocoamidopropyl betaine) | |
| Galguard Trident S (Benzoic acid, capryloyl | 1 |
| glycine and undecylenoyl glycine) | |
| Water Lily- 12366 (oil-soluble fragrance) | 1 |
| Analysis of concentrate | |
| Appearance | Pearly |
| pH | 5.8 |
| Viscosity cps | 790 cps |
| Solids content % | 45% |
| Analysis after four times dilution | |
| Appearance | Pearly |
| pH | 5.5 |
| Viscosity cps at 25° C. | 1800 cps |

We claim:

1. A concentrated reconstitutable liquid composition comprising of:
a. blend of sodium lauroyl methyl taurate and sodium lauroyl methyl isethionate, in the molar ratio of 1:2 and with solids content of minimum 40%;
b. cocamidopropyl betaine with solids content of minimum 40%;
c. 1.0% to 2.5% by weight of total composition, an antimicrobial preservative;
d. 0.5% to 5% by weight of total composition, a cosmetically and pharmaceutically acceptable auxiliaries; and
wherein, the blend of sodium lauroyl methyl taurate and sodium lauroyl methyl isethionate is synthesized using lauroyl chloride;
wherein the ratio of a: b:: 5.0 to 6.0: 3.5 to 4.5 by weight; and the said composition is without any additionally added fatty alcohol or fatty acid as a structurant;
wherein the said composition when diluted 3-5 times, by weight with water to afford a liquid cleansing formulation with pH ranging from 5.0 to 6.0 and viscosity ranging from 2,000 to 10,000 cps at 25° C.

2. The concentrated reconstitutable liquid composition as claimed in claim 1, wherein antimicrobial preservative is selected from one or more benzoic acid, sorbic acid, dehydroacetic acid, undecylenoyl glycine, capryloyl glycine, phenoxy ethanol and/or mixture thereof.

3. The concentrated reconstitutable liquid composition as claimed in claim 1, wherein cosmetically or pharmaceutically acceptable auxiliaries are selected from one or more hair care actives, skin care actives, water-soluble dry herbal extracts, vitamins, chelating agents, inorganic salts, moisturizing agents, soothing agents, nourishing agents, conditioners, colors and fragrances.

4. The concentrated reconstitutable liquid composition as claimed in claim 1, that upon dilution with water afford end-formulation selected from handwash, facewash, bodywash or a shampoo.

5. A process for preparing a concentrated reconstitutable liquid composition comprising:
a. an aqueous blend of sodium lauroyl methyl taurate and sodium lauroyl methyl isethionate;
b. an aqueous solution of cocamidopropyl betaine;
c. 1.0% to 2.5% by weight of total composition, an antimicrobial preservative;
d. 0.5% to 5% by weight of total composition, a cosmetically and pharmaceutically acceptable auxiliaries; and
wherein, the blend of sodium lauroyl methyl taurate and sodium lauroyl methyl isethionate is synthesized using lauroyl chloride in a single reaction vessel;
wherein the ratio of sodium lauroyl methyl taurate and sodium lauroyl methyl isethionate is 1:2;
wherein the solid content of the component a. and the component b. is greater than 40%;
wherein the ratio of a: b:: 5.0 to 6.0: 3.5 to 4.5 by weight; and
wherein the said composition when diluted 3-5 times, by weight, with water, affords a liquid cleansing formulation with pH ranging from 5.0 to 6.0 and viscosity ranging from 2,000 to 10,000 cps at 25° C.
wherein the process comprises the steps of:
i) preparing a 40% to 45% solid content blend of sodium lauroyl methyl taurate and sodium lauroyl methyl isethionate;
ii) preparing cocamidopropyl betaine with solid content of 40% to 45%; and
iii) mixing the components of step i) and step ii) along with an antimicrobial preservative and a cosmetically or pharmaceutically auxiliaries.
